(19) United States
(12) Patent Application Publication
(10) Pub. No.: US 2025/0170272 A1
(43) Pub. Date: May 29, 2025

(54) POLYOMAVIRAL GENE DELIVERY VECTOR PARTICLE COMPRISING A NUCLEIC ACID SEQUENCE ENCODING A PHOSPHATASE ACTIVITY-POSSESSING POLYPEPTIDE

(71) Applicant: AMARNA HOLDING B.V., CH LEIDEN (NL)

(72) Inventors: Ferdy Rudolph Van Diemen, CH LEIDEN (NL); Miguel Garcia Toscano, CH LEIDEN (NL); Simon Delphin Kool, CH LEIDEN (NL); Markwin Paul Velders, CH LEIDEN (NL); Petrus Theodorus De Haan, CH LEIDEN (NL)

(21) Appl. No.: 18/834,387

(22) PCT Filed: Feb. 9, 2023
(86) PCT No.: PCT/NL2023/050057

§ 371 Date: Jul. 30, 2024

(30) Foreign Application Priority Data
Feb. 15, 2022 (NL) 2030945

(51) Int. Cl. A61K 48/00; A61K 9/00; A61K 38/17; A61K 38/46; A61P 27/02; C12N 15/86

(52) U.S. Cl. CPC A61K 48/005; A61K 9/0048; A61K 38/179; A61K 38/465; A61K 48/0075; A61P 27/02; C12N 15/86; C12Y 301/03001; C12N 2710/22043

(57) ABSTRACT

The present invention relates to a polyomaviral gene delivery vector particle comprising a nucleic acid sequence encoding a phosphatase activity-possessing polypeptide. In particular the present invention relates to a polyomaviral gene delivery vector particle comprising a nucleic acid sequence encoding an alkaline phosphatase activity-possessing polypeptide, and/or further comprising a nucleic acid sequence encoding a growth factor protein, such as insulin and/or insulin-like growth factor or precursors thereof. The present invention further relates to a polyomaviral gene delivery vector particle according to the present invention for use in a method of treatment by removal or inactivation of damage-associated molecular patterns at the site of the inflammation in a subject and a composition comprising the polyomaviral gene delivery vector particle of the present invention.

### TECHNICAL FIELD

The present invention relates to a polyomaviral gene delivery vector particle. The present invention further relates to a polyomaviral gene delivery vector particle according to the present invention for use in a method of treatment or prophylactic treatment of inflammation in a subject, e.g. by removal or inactivation of damage-associated molecular patterns at the site of the inflammation in a subject. Further, the present invention relates to a composition comprising the polyomaviral gene delivery vector particle of the present invention.

### BACKGROUND

Autoimmune diseases and allergies are associated with chronic or flaring inflammation and immunity to self-proteins or non-self-proteins named self-antigens or allergens respectively. To date autoimmune diseases and allergies cannot be cured. Symptoms can be masked or alleviated by general suppression of the patients' immune system. Long term use of general immunosuppressants is associated with often severe adverse effects, such as an increased susceptibility to infection with pathogens and development of other immunity-related diseases including cancer, inflammatory, degenerative, dystrophic, autoimmune diseases and allergies. There is therefore an urgent need for novel treatments for autoimmune diseases and allergies that leave the patients' immune system intact.

Inhibition of the chronic or flaring inflammation is an attractive approach to stop inflammatory, degenerative, dystrophic, autoimmune tissue destruction and allergic reactions. Inflammation can be inhibited by restoring the immune tolerance to the primary self-antigens (pSAgs) or allergens in patients with inflammatory, degenerative, dystrophic, autoimmune diseases or allergies respectively. To date however, tolerization approaches to self-antigens or allergens have been poorly effective. In addition, for most of the autoimmune diseases the pSAgs have so far not been identified.

An alternative approach that acts at the innate immunity level to treat autoimmune diseases for which the pSAgs have not been identified is to halt the disease progression by removal of alarm molecules named damage-associated molecular patterns (DAMPs) released from damaged cells. Extracellular adenosine triphosphate (ATP) released from pyroptotic cells is one of the most potent DAMPs. Extracellular ATP drives inflammation, tissue damage and mortality (Cauwels, Anje, et al. “Extracellular ATP drives systemic inflammation, tissue damage and mortality.” Cell death & disease 5.3 (2014): e1102-e1102).

An attractive approach to remove extracellular ATP is its conversion into adenosine diphosphate (ADP), monophosphate (AMP) or adenosine (ADO) by phosphatases present at the site of inflammation. Removal of extracellular ATP molecules will prevent further activation of innate and adaptive immune responses that attack and destroy cells of the affected tissue and will slow down or stop autoimmune disease progression.

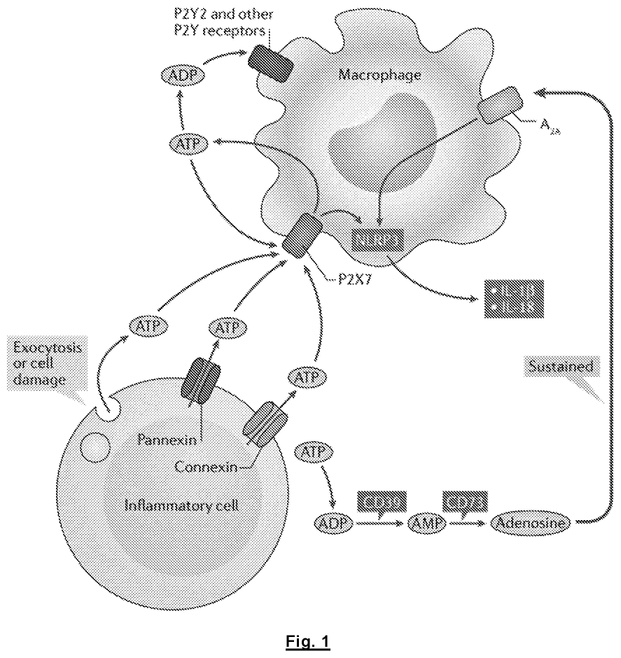

Extracellular ATP is perceived by membrane-bound and G protein-linked P2 receptors (see also: FIG. 1). After binding, mitochondria start to produce excessive ROS, cytoplasmic Nod-like receptor protein 3 (NLRP3) proteins assemble into inflammasomes and a local inflammatory response is initiated. Extracellular ATP is degraded by 2 membrane-bound ectonucleotidases, CD39 (ecto-apyrase) and CD73 (ecto-5′-nucleotidase) into adenosine (ADO). Both nucleotidases are present at the surface of T regulatory cells (Tregs) to prevent activation of immunity cells. Where extracellular ATP is highly proinflammatory, its dephosphorylated derivatives ADP, AMP and ADO perceived by membrane-bound P1 receptors, counteract inflammation and promote the restoration of immune tolerance.

United States patent application US 2009/0130092 A1 discloses a method of treating or preventing immunoinflammatory, thrombotic or ischemic disorders in a subject by inhibiting leukocyte infiltration into a site which comprises administering to the subject an effective amount of ecto-apyrase and/or ecto-5′-nucleotidase proteins at the site of inflammation. One of the disadvantages of such a method is that the proteins have a relatively short half-life in vivo. This implies that the patients have to be administered repeatedly and life-long with the proteins to keep the inflammation at a low level.

The problem of the short half-life in vivo of the ecto-apyrase and/or ecto-5′-nucleotidase proteins may be circumvented by using a viral gene delivery vector to express ecto-apyrase and/or ecto-5′-nucleotidase proteins (soluble forms or fusion proteins thereof) at the site of inflammation.

Among the viral gene delivery vectors currently used for these purposes, lentiviral (LV) and adeno-associated viral (AAV) vectors are the most widely used. For both vectors it has been shown that such replication-defective viral vectors are non-immunogenic or tolerogenic in hosts that are naive to the cognate virus.

LV vectors integrate randomly in the host genome and thus increase the risk of insertional mutagenesis. Moreover, LV vector particles are rapidly degraded when administered in vivo. For this reason, LV vectors are mainly used for the ex vivo transduction of leukocytes and/or their progenitors to treat blood-related genetic disorders, lysosomal storage diseases or cancer.

AAV vectors, are mainly used for in vivo gene therapies. For example, International patent application WO 2014/003553 A1 discloses the use of AAV vectors to express ecto-apyrase and/or ecto-5′-nucleotidase proteins or fusion proteins thereof at the site of inflammation. United States patent application US 2015/0190481 A1 and Antonioni (Antonioli, Luca, et al. “CD39 and CD73 in immunity and inflammation.” Trends in molecular medicine 19.6 (2013): 355-367) deal with ecto-apyrase- and/or ecto-5′-nucleotidase-encoding sequences in viral gene delivery vectors to treat an inflammatory disease. The ecto-apyrase- and/or ecto-5′-nucleotidase proteins remain bound to the cell surface of the transduced cells, which results in a very low phosphatase activity in the inflamed tissues and limited therapeutic benefit. Matsumoto et al., used an AAV vector encoding the secreted tissue non-specific alkaline phosphatase (TNALP) to restore the TNALP activity in an animal model for human infantile hypophosphatasia with the aim to dampen the characteristic disease symptoms such as epileptic seizures in the treated animals (Matsumoto, Tae, et al. “Rescue of severe infantile hypophosphatasia mice by AAV-mediated sustained expression of soluble alkaline phosphatase.” Human gene therapy 22.11 (2011): 1355-1364).

However, before receiving a treatment with an AAV vector, the majority of the human population already encountered wild type AAV together with its helper virus (adenovirus, causing the common cold) and thus already developed a strong humoral and cellular immune memory for the AAV capsid proteins. Clinical studies using recombinant AAV vectors revealed that administration of vector particles elicit innate and adaptive immune responses against the viral and transgene-encoded proteins in the vast majority of treated patients leading to decreasing expression levels of the therapeutic transgenes over time and elimination of the transduced cells from the body compromising re-administration of the vector. The few treated patients that showed long term transgene expression most likely have never been infected with AAV and thus were immunologically naïve to the vector used in the study. For these reasons, the efficacy of AAV vector-based in vivo gene therapies is limited.

### DETAILED DESCRIPTION

Given the above, there is thus a need for a novel treatment for autoimmune diseases and/or allergies that has a high efficacy and/or having a reduced patient discomfort, while leaving the patients' immune system intact. The present invention provides hereto, in a first aspect, a polyomaviral gene delivery vector particle comprising a nucleic acid sequence encoding a phosphatase activity-possessing polypeptide. It was found that the polyomaviral gene delivery vector particle of the present invention effectively protects cells from undergoing pyroptosis after exposure to ATP. Cells transduced with polyomaviral gene delivery vector particles of the present invention are effectively protected from ATP-induced pyroptosis.

The term “viral gene delivery vector particle” as used herein refers to a transduction-competent viral particle comprising capsid proteins exposed on the surface of the particle surrounding a viral vector genome with an incorporated transgene construct.

The term “transgene construct” as used herein refers to a nucleic acid sequence incorporated in a viral gene delivery vector particle, encoding a polypeptide or protein.
The term “polyomaviral gene delivery vector particle” as used herein refers to viral vector particles derived from a polyomavirus. Polyomaviruses (family: polyomaviridae) naturally infect primarily mammals and birds where they cause chronic asymptomatic infections. Each polyomaviral species strictly replicates in its natural host. Of the about 120 polyomavirus species known to date, only 14 of them are known to infect humans. These polyomaviruses are referred to as human polyomaviruses. The other polyomaviruses do not infect humans and are referred to as non-human polyomaviruses. This implies that humans do not have an immune memory for non-human polyomaviruses. As a result, the polyomaviral gene delivery vector particles of the present invention will be non-immunogenic in humans. Administration of humans with polyomaviral gene delivery vector particles of the present invention will result in the induction of an immune tolerance response directed to the viral proteins and proteins encoded by the transgene construct. Typically polyomaviral gene delivery vectors are safe, highly efficient for gene delivery and non-immunogenic/tolerogenic in humans.

In particular, the present invention relates to a polyomaviral gene delivery vector particle comprising a nucleic acid sequence encoding a phosphatase activity-possessing polypeptide, wherein the polyomaviral gene delivery vector particle comprises a replication-defective polyomaviral gene delivery vector particle. Thus providing a gene delivery vector particle that is defective for one or more functions that are essential for viral genome replication or synthesis and assembly of viral particles.

In a preferred embodiment of the present invention, the polyomaviral gene delivery vector particle may be derived from a primate polyomavirus, preferably a simian polyomavirus, such as macaque polyomavirus Simian Virus 40 (SV40). It was found that SV40 vectors, preferably replication-defective SV40 vectors, are an attractive alternative to AAV vectors for clinical gene therapy. SV40 is a polyomavirus with icosahedral capsids of 45 nanometres in size containing a 5.25 kb long circular double-stranded DNA genome. The virus strictly replicates in its natural host, macaques, where it causes chronic asymptomatic infections. SV40 particles enter infected cells via the caveolar-endosomal route, but in contrast to other viruses are able to avoid the lysosomal compartment, thereby evading exposure to the host immune system.

Replication-defective SV40 vectors have been generated by deleting the coding region of the two early non-structural proteins named large T antigen (LTag) and small T antigen (STag) giving 2.7 kb of space for cloning the transgene encoding the therapeutic protein or RNA. SV40 vectors transduce a wide range of cell types in vivo and their therapeutic potential has been demonstrated in animal models of human disease. Since humans are immunologically naive for SV40, replication-defective SV40 vectors are non-immunogenic and tolerogenic when applied in clinical settings. The non-immunogenicity in humans and capacity to induce immune tolerance to transgene proteins render SV40 vectors highly attractive for use in gene therapies (Toscano, M. G., et al. “Generation of a vero-based packaging cell line to produce SV40 gene delivery vectors for use in clinical gene therapy studies.” Molecular Therapy—Methods & Clinical Development 6 (2017): 124-134; Toscano, M. G., and De Haan, P. “How simian virus 40 hijacks the intracellular protein trafficking pathway to its own benefit . . . and ours.” Frontiers in Immunology 9 (2018): 1160; Vera, M. and Fortes, P. “Simian virus-40 as a gene therapy vector.” DNA and cell biology 23.5 (2004): 271-282).

The term “phosphatase activity-possessing polypeptide” as used herein refers to a phosphatase or active derivative thereof having phosphatase activity, i.e. referring to a polypeptide (e.g. protein) that is able to dephosphorylate compounds. The polypeptide of the present invention may be a secreted form of a phosphatase activity-possessing polypeptide. Particular good results are observed by selecting a polypeptide being an alkaline phosphatase activity-possessing polypeptide. The polypeptide of the present invention may be selected from the group of alkaline phosphatases as being found across a multitude of organisms, prokaryotes and eukaryotes alike, with the same general function but in different structural forms suitable to the environment they function in. Preferably, alkaline phosphatase activity-possessing polypeptide is selected from the group of mammalian, such as human, alkaline phosphatases or active derivatives thereof.

In a first aspect of the present invention, the invention provides the insight that the disadvantage of the use of ecto-apyrase and/or ecto-5′-nucleotidase for inhibiting inflammation is that the phosphatase activity of ecto-apyrase and ecto-5′-nucleotidase is low compared to that of alkaline phosphatases in particular. By providing a polyomaviral gene delivery vector particle able to express alkaline phosphatases, such as the four membrane-bound alkaline phosphatases named germ cell alkaline phosphatase (GCALP; SEQ ID NO: 1), placental alkaline phosphatase (PALP; SEQ ID NO: 2), Intestinal alkaline phosphatase (IALP; SEQ ID NO: 3) and tissue non-specific alkaline phosphatase (TNALP; SEQ ID NO: 4), a highly effective autoimmune and/or allergy therapy can be provided. It was found that the phosphatase activity of the alkaline phosphatases in particular is higher than that of ecto-apyrase and ecto-5′-nucleotidase. In other words, the term “alkaline phosphatase activity-possessing polypeptide” as used herein refers to alkaline phosphatases or derivatives thereof having a higher phosphatase activity compared to ecto-apyrase and ecto-5′-nucleotidase.

In particular, it was found that a polyomaviral gene delivery vector particle comprising a nucleic acid sequence encoding a secreted alkaline phosphatase or active derivative thereof resulted in the most effective method to protect cells from undergoing pyroptosis after exposure to ATP. In a preferred embodiment, the secreted alkaline phosphatase or active derivative thereof is selected from a secreted variant of human PALP (SPALP), a secreted variant of human IALP (SIALP), a secreted variant of human GCALP (SGCALP), a secreted variant of human TNALP (STNALP) or active derivatives thereof. More preferably, the alkaline phosphatase activity-possessing polypeptide comprises a nucleic acid sequence encoding SPALP.

As the present invention relates to a polyomaviral gene delivery vector particle comprising a nucleic acid sequence encoding an alkaline phosphatase activity-possessing polypeptide, it is noted that also alkaline phosphatase derivates may be used to possess phosphatase activity. As a consequence, although the nucleic acid sequence preferably comprises a nucleic acid sequence (at least partially) identical to SEQ ID NO: 5 (SPALP), the nucleic acid sequence may have a substantial nucleic acid sequence homology to the target nucleic acid sequence (encoding a phosphatase activity-possessing polypeptide). Preferably the nucleic acid sequence encoding the phosphatase activity-possessing polypeptide comprises (at least partially) a nucleic acid sequence having at least 70, 80, 90, 91, 92, 93, 94, 95, 96, 97, 98, 99 or 100% sequence identity to the amino acid sequence SEQ ID NO: 5 (SPALP).

The term “nucleic acid sequence homology” as used herein denotes the presence of homology between two polynucleotides. Polynucleotides have “homologous” sequences when either a sequence of nucleotides in the two polynucleotides is the same or when a sense sequence of the one and an antisense sequence of the other polynucleotide is the same when aligned for maximum correspondence. Sequence comparison between two or more polynucleotides is generally performed by comparing portions of at least two sequences over a comparison window to identify and compare local regions of sequence similarity. The comparison window is generally from about 20 to 200 contiguous nucleotides in length.

The “percentage of sequence identity” for polynucleotide sequences of the invention, such as at least 70, 80, 90, 91, 92, 93, 94, 95, 96, 97, 98, 99 or 100% sequence identity may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence identity (also referred to as sequence homology). Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul S. F. et al., Basic local alignment search tool. J. Mol. Biol. 215:403-410, 1990; Altschul S. F. et al. Gapped BLAST and PSI-BLAST: a new generation of protein database search programs. Nucl. Acids Res. 25:3389-3402, 1997) and ClustalW programs both available on the internet. Other suitable programs include GAP, BESTFIT and FASTA in the Wisconsin Genetics Software Package (Genetics Computer Group (GCG), Madison, WI, USA).

The homology between nucleic acid sequences may be determined with reference to the ability of the nucleic acid sequences to hybridise to each other upon denaturation (e.g., under conditions of 400 mM NaCl, 40 mM PIPES pH 6.4, 1 mM EDTA, at a temperature of 50 degrees Celsius to 65 degrees Celsius and hybridisation for 12-16 hours, followed by washing) (Molecular Cloning: a Laboratory Manual: 2nd edition, Sambrook et al., Cold Spring Harbor Laboratory Press, 1989 or Current Protocols in Molecular Biology, Second Edition, Ausubel, F. et al. eds., John Wiley & Sons, 1992).

Generally speaking, those skilled in the art are well able to construct polyomaviral gene delivery vectors and design protocols suitable for use in the present invention, i.e. expressing phosphatase activity-possessing polypeptides. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook, J. et al., Cold Spring Harbor Laboratory Press, 1989.

In an embodiment of the present invention the polyomaviral gene delivery vector particle may consist essentially of a nucleic acid sequence encoding an alkaline phosphatase activity-possessing polypeptide. In other words, the polyomaviral gene delivery vector particle of the present invention may be substantially free of or may not comprise a nucleic acid sequence encoding an ectonucleotidase, such as a secreted ectonucleotidase. It was found that the phosphatase activity of ectonucleotidases is too low compared to alkaline phosphatases in particular for inhibiting inflammation. In particular, the polyomaviral gene delivery vector particle of the present invention may be substantially free of or may not comprise a nucleic acid sequence encoding ecto-apyrase and ecto-5′-nucleotidase.

In a second aspect of the present invention, the invention relates to a polyomaviral gene delivery vector particle comprising a nucleic acid sequence encoding a phosphatase activity-possessing polypeptide, wherein the particle further comprises a nucleic acid sequence encoding a growth factor protein. A growth factor protein is a naturally occurring protein capable of stimulating cell proliferation, wound healing and cellular differentiation. Examples of growth factor proteins are adrenomedullin, angiopoietin, autocrine motility factor, bone morphogenetic proteins, the ciliary neurotrophic factor family, colony-stimulating factors, epidermal growth factor, ephrins, erythropoietin, fibroblast growth factors, the GDNF family of ligands, growth differentiation factor-9, hepatocyte growth factor, hepatoma-derived growth factor, insulin, insulin-like growth factors (IGFs), interleukins, keratinocyte growth factor, migration-stimulating factor, macrophage-stimulating protein, myostatin, neuregulins, neurotrophins, placental growth factor, platelet-derived growth factor, renalase, T-cell growth factor, thrombopoietin, transforming growth factors, Tumor necrosis factor-alpha and vascular endothelial growth factor.

It was found that the phosphatases expressed by the polyomaviral gene delivery vector particle of the present invention are particularly active in combination with a nucleic acid sequences encoding insulin and/or IGFs including insulin-like growth factor 1 (IGF1; SEQ ID NO: 6) and insulin-like growth factor 2 (IGF2; SEQ ID NO: 7).

Preferably the nucleic acid sequence encoding insulin and/or IGFs or precursors thereof comprises a nucleic acid sequence encoding a secreted form of insulin and/or IGFs or precursors thereof. Also, similar to the nucleic acid sequence encoding the phosphatase activity-possessing polypeptide, the nucleic acid sequence encoding insulin and/or IGFs or precursors thereof comprises (at least partially) a nucleic acid sequence having at least 70, 80, 90, 91, 92, 93, 94, 95, 96, 97, 98, 99 or 100% sequence identity to the amino acid sequence SEQ ID NO: 6 (IGF1) or SEQ ID NO: 7 (IGF2).

In an embodiment of the present invention the polyomaviral gene delivery vector particle may comprise a first nucleic acid molecule comprising the nucleic acid sequence encoding a phosphatase activity-possessing polypeptide and a second nucleic acid molecule comprising the nucleic acid sequence encoding insulin and/or IGFs or precursors thereof.

In an alternative embodiment of the present invention the polyomaviral gene delivery vector particle may comprise a nucleic acid molecule comprising the nucleic acid sequence encoding a phosphatase activity-possessing polypeptide and the nucleic acid sequence encoding insulin and IGFs or precursors thereof.

In a further embodiment of the present invention, the polyomaviral gene delivery vector particle may comprise a nucleic acid sequence encoding a growth factor-binding protein, such as an insulin growth factor-binding protein (IGFBP, including IGFBP1, IGFBP2, IGFBP3, IGFBP4, IGFBP5, IGFBP6 and IGFBP7) or precursors thereof.

The term “nucleic acid molecule” as used herein includes both DNA molecules, such as cDNA or genomic DNA, and RNA molecules, such as mRNA, and analogues of the DNA or RNA generated using nucleotide analogues. The nucleic acid molecule can be single-stranded or double-stranded, but preferably is double-stranded DNA.

In a third aspect of the present invention, the invention relates to the polyomaviral gene delivery vector particle of the present invention for use in a method of treatment or prophylactic treatment (e.g. prevention) of inflammatory, degenerative, dystrophic, autoimmune diseases and/or allergies. In an embodiment, the invention relates to the polyomaviral gene delivery vector particle of the present invention for use in a method of treatment or prophylactic treatment by removal or inactivation damage-associated molecular patterns at the site of inflammation in a subject. In particular, the present invention may relate to a polyomaviral gene delivery vector particle according to the present invention for use in a method of treatment or prophylactic treatment by removal of extracellular ATP in a subject. For example, the polyomaviral gene delivery vector particle of the present invention may be used in a method of treating a subject with inflammatory, degenerative, dystrophic, autoimmune diseases and/or allergies.

Examples of diseases suitable for treatment with the polyomaviral gene delivery vector particle of the present invention may include sporadic retinal degenerative diseases such as age-related macular degeneration (AMD), glaucoma and diabetic retinopathy (DR). For example, AMD is associated with chronic inflammation of macular retinal pigment epithelium (RPE) cells generally followed by choroidal hypervascularization beneath the inflamed RPE cells resulting in irreversible T cell-mediated macular damage and a loss of central vision and blindness. It has been reported that ATP released from RPE, neuronal and endothelial cells in the retina plays an important role in the development of AMD, glaucoma and DR respectively (Ventura, Ana Lucia Marques, et al. “Purinergic signaling in the retina: From development to disease.” Brain research bulletin 151 (2019): 92-108).

Other examples of diseases suitable for treatment with the polyomaviral gene delivery vector particle of the present invention may include chronic obstructive pulmonary disease (COPD), asthma, arthritis, non-alcoholic fatty liver disease (NAFLD), non-alcoholic steatohepatitis (NASH), liver cirrhosis and rheumatoid and osteoarthritis.

In order to treat subjects polyomaviral gene delivery vector particles are administered to the subject. Preferably the polyomaviral gene delivery vector particles are administered at the site of inflammation, e.g. by intravenous, intrasynovial, intrapulmonary, intravitreal or subretinal injection. The polyomaviral gene delivery vector particles may be administered to any mammal naive for polyomaviruses. However, preferably, polyomaviral gene delivery vector particles are administered to a subject wherein the subject is human.

In a fourth aspect of the present invention, the invention relates to a composition comprising an effective amount of polyomaviral gene delivery vector particles of the present invention. The invention further relates to a composition comprising an effective amount of polyomaviral gene delivery vector particles for use as a medicament.

In an embodiment of the present invention, the composition may further comprise insulin, IGF such as IGF1 and IGF2 and/or IGFBPs or precursors thereof. It was found that the phosphatases expressed by the polyomaviral gene delivery vector particle of the present invention are particularly active in combination with insulin, IGF such as IGF1 and IGF2 and/or IGFBPs or precursors thereof.

The term “effective amount” as used herein refers to a dosage which is sufficient in order for the treatment of the subject to be effective compared with no treatment. The term “effective amount” refers to an amount having a desired therapeutic effect, i.e. “a therapeutically effective amount”.

The term “therapeutically effective amount” as used herein refers to an amount sufficient to cure, alleviate or partially arrest the clinical manifestations of a given disease and its complications. An amount adequate to accomplish this is defined as “therapeutically effective amount” or “effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In respect to the present invention, the (therapeutically) effective amount may be expressed in number of polyomaviral gene delivery vector particles. As the effective amount highly depends on the actual number of vector particles, one could also determine the actual number of vector particles to be administered to the subject.

### Experiments

The feasibility of a gene delivery vector derived from Simian virus 40 (SV40), hereinafter referred to as “SVec vector”, encoding a secreted variant of the human placental alkaline phosphatase (SPALP), hereinafter referred to as “SVSPALP”, for its use in treating AMD after intravitreal administration was investigated.

The gene delivery vectors used were prepared using the methods disclosed in International patent application WO 2010/122094 A1 and Toscano (Toscano, M. G., et al. “Generation of a vero-based packaging cell line to produce SV40 gene delivery vectors for use in clinical gene therapy studies.” Molecular Therapy—Methods & Clinical Development 6 (2017): 124-134).

### In Vitro AMD Disease Model

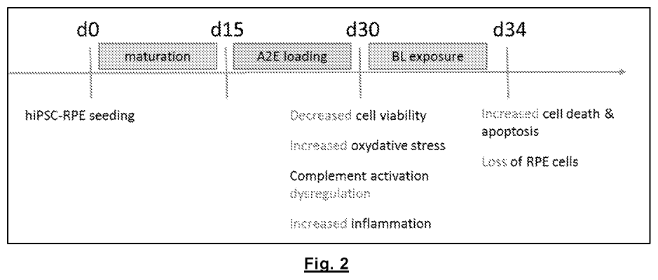

AMD is an idiopathic retinopathy for which established animal disease models have not become available. An in vitro cell-based model developed by Phenocell SAS was used to obtain proof-of-principle of the SVec-based therapy to treat dry AMD. An overview of the in vitro Dry AMD model developed by Phenocell SAS is depicted in FIG. 2.

The model relies on human induced pluripotent stem cells that differentiate (maturation) into RPE-like cells after treatment with the myosin inhibitor blebbistatin (Maruotti, J., et al. “A simple and scalable process for the differentiation of retinal pigment epithelium from human pluripotent stem cells.” Stem cells translational medicine 2.5 (2013): 341-354; Frank, D. and Vince J. E. “Pyroptosis versus necroptosis: similarities, differences, and crosstalk.” Cell Death & Differentiation 26.1 (2019): 99-114).

After this maturation stage of 15 days the RPE-like cells are incubated with the retinal photosensitizer A2E for another 15 days (A2E loading). A2E is a retinoid constituent of lipofuscin that as a DAMP induces oxidative stress resulting in the intracellular production of reactive oxygen species (ROS).

Blue light exposure for 4 days further enhances the generation of ROS and induces pyroptosis of the RPE-like cells. To gain insight into the inflammatory processes that underlie blue light-induced pyroptosis of RPE-like cells, total ROS production and cell death due to pyroptosis are determined at day 34. In addition, at day 34 the expression of proinflammatory genes such as IL-1b, IL-6, IL-8, IL-18, TNF-α and IFN-γ, Caspase 1, C3, IGF-1, GFAP are measured by PCR or ELISA.

### Efficient and Long-Term Gene Transduction of Mouse Retinal Cells Using GFP-Expressing SV40 Vectors

To demonstrate that SV40-derived vectors can transduce retina cells of C57BL/6 mice an SV40 vector expressing the jellyfish green fluorescent protein (GFP) (SVGFP) was used. The expression of the GFP cDNA is driven by the SV40 early promoter. Two different doses of vector particles (vector genomes, VG), ranging from 10⁷ to 10⁸ VG and two different routes of delivery, subretinal and intravitreally, were tested. The vector administrations were performed in one eye and the counter eye was used as control, in which an equal volume of PBS was injected. One month later GFP expression was determined by fluorescence fundoscopy. The GFP fluorescent signal was detected in the retinas of mice that were administered in the subretinal space with both SVGFP vector doses. This signal was widely spread over the eye of the mouse. Also, the GFP fluorescent signal was detected in the retina of mice administered intravitreally with the high vector dose, but not in those administered with the low dose.

GFP fluorescence in the retina of mice that received the high vector dose in the subretinal space was monitored for up to 12 months using fundoscopy. The GFP fluorescent signal was detected across all measurements taken up to 12 months. Qualitatively, the spread and intensity of the fluorescence was maintained at similar levels until month 10. At the end of the experiment, the intensity of the fluorescent signal diminished slightly, though remained detectable.

To find out which cell types were preferentially targeted after delivery of SVGFP particles, either when subretinally or intravitreally administered, the eyes of the mice were processed to obtain cryo-sections to evaluate them under confocal or structured illumination (Apotome) microscopy. In all cases, DAPI staining was used to reveal the tissue structure. First, an anti-GFP antibody was used to verify that the signal acquired in the 488 nm channel that detects GFP fluorescence belonged to the signal of the GFP protein. The presence of GFP in the mouse retina was observed. The location of the GFP signal indicated that retinal pigmented epithelium (RPE) cells were predominantly transduced. This was confirmed by using an antibody against the RPE65 protein that labels the RPE cell layer. It was observed that GFP was not found in the photoreceptor layer. However, both photoreceptor and Müller cells are susceptible of being transduced by SVGFP, although at the tested dose the efficiency was testimonial.

Second, the slides obtained from mice receiving the intravitreal administration of SVGFP particles were also single-labelled with antibodies to identify the RPE and photoreceptor cell layer. The GFP signal was not present in the RPE nor photoreceptor cell layers, but the majority of transduced cells seem to be microglia. Microglia are widely distributed within the outer and inner nuclear layer and close to the ganglion cell layer. GFP expression was also found in cells of the ganglion cell layer. Electroretinography of the injected mouse eyes revealed that the transduction of retinal cells does not impair their visual function.

### Biodistribution Analysis of the SVGFP Vectors in Mice

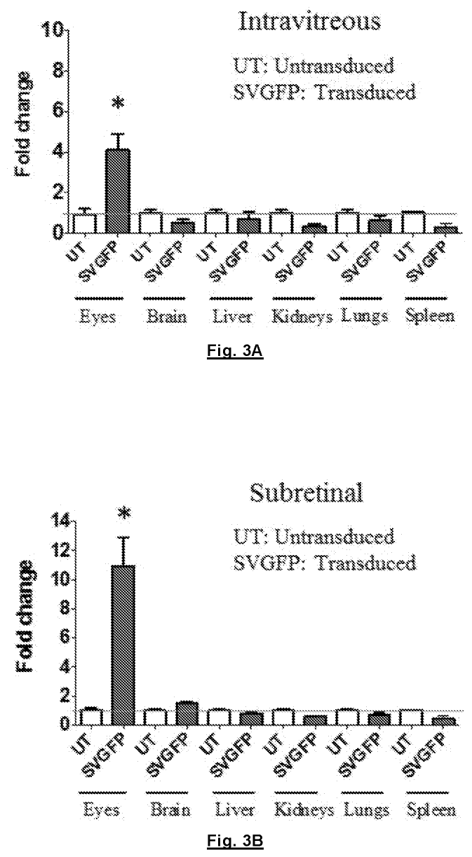

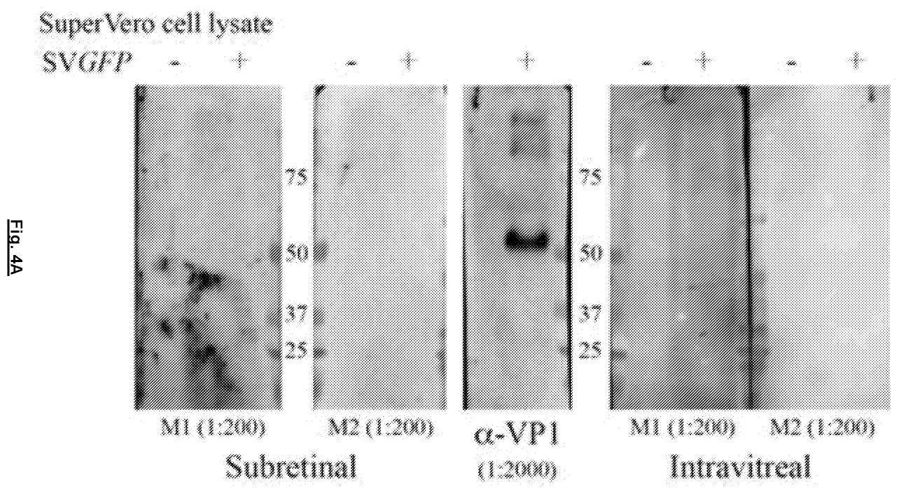

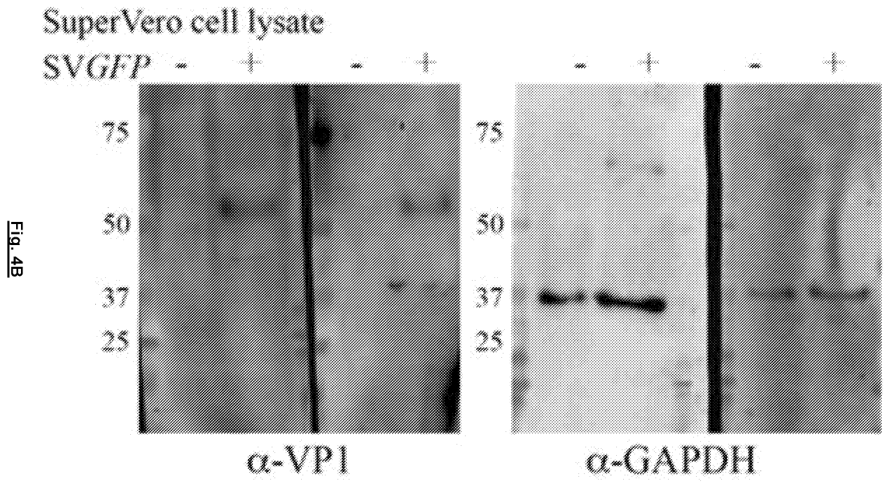

To evaluate whether SVGFP particles are capable of transducing other cell types outside of the retina after subretinal or intravitreal delivery, genomic DNA from different tissues were isolated and a qPCR was performed using primers specific for a sequence of the late region of the SV40 genome. The same setup was used that is used to determine the number of vector particles. Thus, as a template genomic DNA isolated from brain, kidneys, lungs, spleen, liver and eyes was used. SVGFP-specific DNA sequences could only be detected in DNA samples isolated from the vector-injected eyes (FIG. 3A). SVGFP particles were not detected in any of the DNA samples isolated from the rest of the tested tissues. A non-significant increase in the presence of SVGFP vector particles was detected in the brain tissue from mice of the subretinal group (FIG. 3B). However, further confocal analysis of immunostained slides prepared from the brain tissue of these mice did not confirm the presence of the vector (data not shown).

### Analysis of Anti-SV40 Neutralizing Antibodies

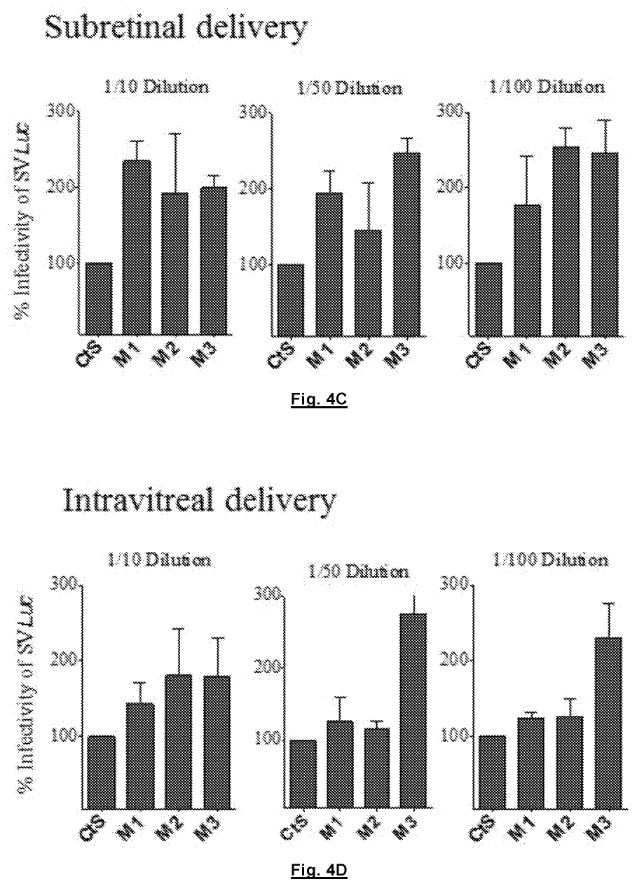

As observed for other gene therapy vectors, the presence or induction of neutralizing antibodies against the vector itself may decrease the transgene expression level and result in elimination of transduced cells from the body. Therefore, it was determined whether after both subretinal and intravitreal administrations, antibodies against the SV40 capsid components could be detected and whether these antibodies possessed neutralizing activity. VP1 is the major capsid protein of the SV40 particle. First, a Western blot was developed using lysates from SuperVero cells (Amarna Therapeutics) transduced with SVGFP as a source of VP1 capsid protein. Non-transduced SuperVero cells were used as a negative control. A commercial antiserum containing antibodies against the VP1 capsid protein was used as a positive control for the detection of VP1 (FIG. 4A, middle lane). Incubation of the membranes containing the SuperVero cell lysates with serum samples collected from both subretinally and intravitreally SVGFP-administered mice did not reveal the presence of VP1 (FIG. 4A). The same membranes were incubated with the VP1 commercial serum or anti-GAPDH antibodies to confirm that the membranes contained the VP1 protein from the SuperVero cell lysates (FIG. 4B).

Second, a neutralizing assay was performed using COS-1 cells, that were transduced at an MOI of 100 with SVLuc particles pre-incubated with different dilutions of sera obtained from subretinally and intravitreally SVGFP-administered animals (FIG. 4C and FIG. 4D). The vector particles were also pre-incubated with dilutions of a serum obtained from an untreated control mouse. A positive control of neutralizing antibody activity is shown in FIG. 4E by using a commercially available rabbit anti-VP1 serum. There was no decrease in the transduction capacity of SV40 vector particles observed when they were pre-incubated with sera obtained from animals transduced with an SV40 vector, indicating that SV40 vector particles do not induce the generation of neutralizing antibodies after intra-ocular administration.

### Analysis of the SV40 Main Capsid Protein VP1 in Target Cells

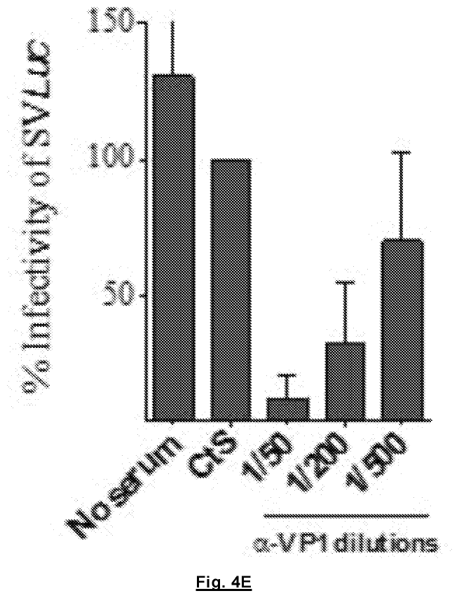

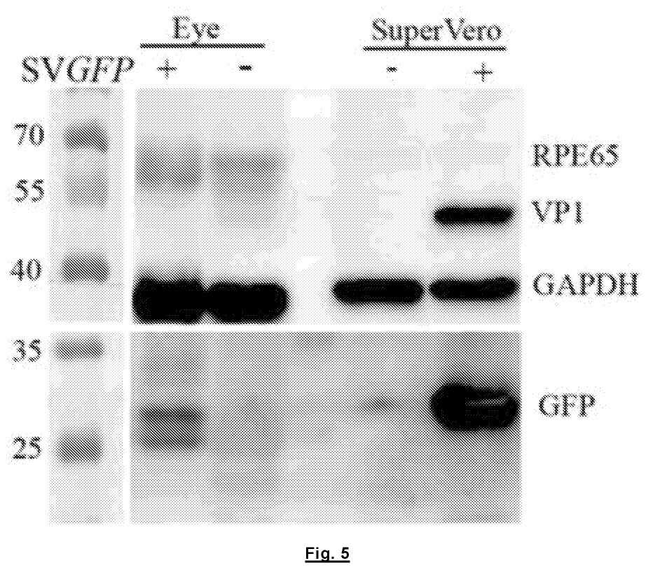

The SV40 early gene encoding the viral structural proteins is exclusively expressed in macaque cells expressing the viral Large T antigen, during the production of vector particles in SuperVero packaging cells (Toscano, M. G., et al. “Generation of a vero-based packaging cell line to produce SV40 gene delivery vectors for use in clinical gene therapy studies.” Molecular Therapy—Methods & Clinical Development 6 (2017): 124-134). In order to obtain a confirmation that the viral structural genes are not expressed in vivo, the presence/absence of the VP1 capsid protein in the eye of the SVGFP-administered mice was determined. Using protein samples obtained from SVGFP-transduced and non-transduced mouse eyes and SuperVero cells transduced with SVGFP as controls, it was confirmed that the VP1 capsid protein was not present in the eyes of mice that received vector particles. Only the transgene protein delivered by the SV40 vector, in this case GFP, was detected in the lysates from the eyes of the treated mice (FIG. 5).

In Vitro Testing of SVec Vectors Encoding Anti-Inflammatory Phosphatases for their Capacity to Inhibit Blue Light-Induced Pyroptosis of RPE Cells

SVec vectors encoding SCD39L4 and SPALP were constructed yielding pSVSCD39L4 (pAM396) and pSVSPALP (pAM397) respectively. Human pluripotent stem cells were nucleofected with pSVGFP DNA and after 30 days GFP expression was measured by fluorescence microscopy. The induced RPE-like cells retain stable GFP expression, indicating that the experimental set-up is valid.

Human pluripotent stem cells were nucleofected with pSVGFP DNA. The cells were subsequently maturated, loaded with A2E and exposed to blue light after 30 days. At 34 days post nucleofection cell death due to pyroptosis was determined. Quercetin protects cells from pyroptosis and was used as a positive control in the experiments. Following A2E loading and blue light exposure there was a strong decrease in the cell survival observed (from 32% in vehicle condition, down to 0.01% in A2E condition). The loss of living cells was significantly counteracted by quercetin treatment (with 15.5% living cells).

Without blue light exposure, the RPE-like cells maintained a healthy phenotype (91.7% living cells), while quercetin exposure rescued cell survival up to 32.5%. Next human pluripotent stem cells were nucleofected with pSVGFP, pSVSCD39L4 (pAM396) or pSVSPALP (pAM397) DNA. The cells were matured, A2E loaded and exposed to blue light and at 34 days post nucleofection the ROS production and cell death was determined. In addition, the production of the production of the pro-inflammatory markers was measured by PCR or ELISA.

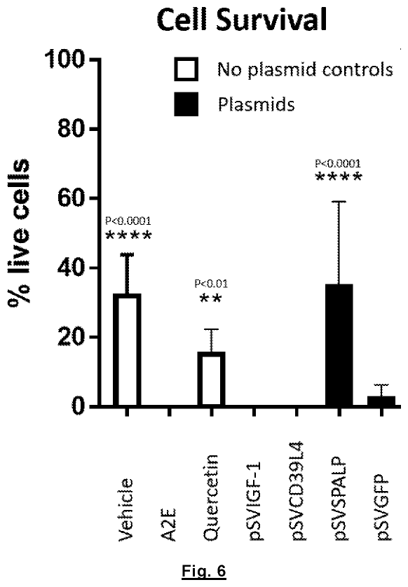

Nucleofection of RPE-like cells with pSVSPALP (pAM397) DNA provides a significant rescue in cell survival after blue light exposure, with 35.2% living cells (slightly above vehicle levels) and even better than that of cells treated with quercetin (15%) (FIG. 6). The ROS measurements and measurements on the production of the proinflammatory markers at day 34 post nucleofection did not reveal any differences between the untreated and nucleofected cells (results not shown). Overall, RPE-like cells containing vector pSVSPALP, but not pSVCD39L4 are effectively protected from pyroptosis after exposure to blue light.

pSVSPALP in Combination with pSVIGF1 Enhances the Therapeutic Effects

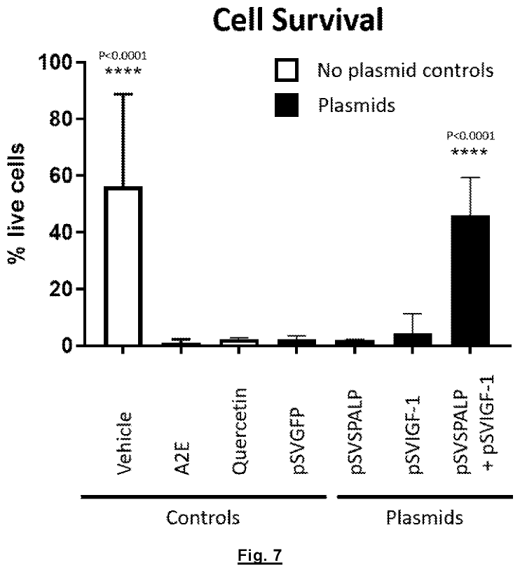

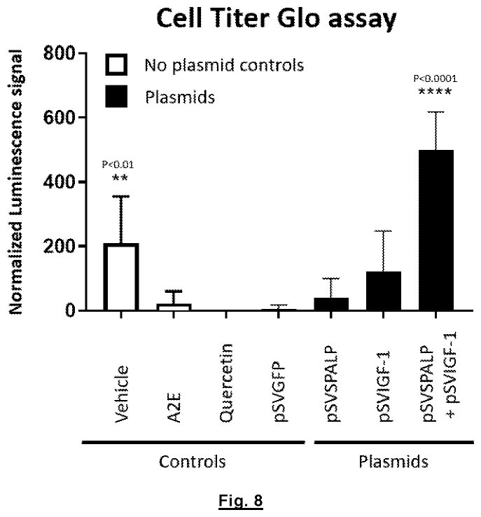

Nucleofection of RPE-like cells with a combination of pSVSPALP and pSVIGF1 DNA provides a significant synergistic rescue in cell survival after blue light exposure at day 41 post nucleofection, with 46% living cells (slightly below vehicle (untreated) control levels) and even better than that of cells treated with the positive control quercetin (FIG. 7). These results could also be confirmed in a second assay in which intracellular ATP is measured as a marker for cell viability (Cell Titer Glo assay, Promega). Here, the combination of pSVSPALP and pSVIGF1 outperformed the single nucleofection of pSVSPALP or pSVIGF1 alone (FIG. 8). Overall, RPE-like cells containing vector pSVSPALP, pSVIGF1 were protected from pyroptosis after exposure to blue light. However, cell cultures of RPE-like cells that received both pSVSPALP and pSVIGF1 were significantly better protected from pyroptosis after exposure to blue light than pSVSPALP or pSVIGF1 individually.

### CONCLUSION

Given the above, it was shown that an SVec vector encoding the jellyfish green fluorescent protein (SVGFP) injected intravitreally to the eyes of mice and GFP expression in the retina and monitored up to one year after administration, resulted in the efficient transduction of ganglion cells and microglia, without any adverse side effects or loss of vision. It was shown that an SVec vector encoding secreted placental alkaline phosphatase (SVSPALP), effectively protects RPE-like cells from pyroptosis after blue light exposure. The protection is most likely based on the fact that SPALP released from the RPE-like cells converts the ATP released from inflamed cells into inactive metabolites ADP and AMP.

Taken together, the above experiment shows that SVec vectors are highly promising gene delivery vehicles for the treatment of ophthalmological diseases. In addition, SVSPALP is a highly promising vector for effectively halting retinal inflammation to not only treat AMD, but also glaucoma, DR and other autoimmune conditions that are associated with local tissue inflammation.

In addition to the above, it was found that a replication-defective SV40 gene delivery vector encoding a soluble form of the human PALP denoted SPALP (SVSPALP) effectively protects Vero cells from undergoing pyroptosis after exposure to ATP. In addition, retinal pigment epithelium cells expressing SPALP encoded by SVSPALP are protected from blue light-induced pyroptosis. The vector was further used in mouse models of age-related macular degeneration, non-alcoholic steatohepatitis, arthritis and asthma to stop inflammation and prevent disease progression.

1. Polyomaviral gene delivery vector particle comprising a nucleic acid sequence encoding a phosphatase activity-possessing polypeptide.

2. Polyomaviral gene delivery vector particle according to claim 1, wherein the polyomaviral gene delivery vector particle comprises a replication-defective polyomaviral gene delivery vector particle.

3. Polyomaviral gene delivery vector particle according to claim 1, wherein the polyomaviral gene delivery vector particle is derived from a primate polyomavirus.

4. Polyomaviral gene delivery vector particle according to claim 1, wherein the nucleic acid sequence encoding a phosphatase activity-possessing polypeptide comprises a nucleic acid sequence encoding a secreted form of a phosphatase activity-possessing polypeptide.

5. Polyomaviral gene delivery vector particle according to claim 1, wherein the nucleic acid sequence encoding a phosphatase activity-possessing polypeptide comprises a nucleic acid sequence encoding an alkaline phosphatase activity-possessing polypeptide or active derivative thereof.

6. Polyomaviral gene delivery vector particle according to claim 5, wherein the nucleic acid sequence encoding an alkaline phosphatase activity-possessing polypeptide comprises a nucleic acid sequence encoding a secreted variant of human placental alkaline phosphatase (PALP), human intestinal alkaline phosphatase (IALP), human germ cell alkaline phosphatase (GCALP), human tissue non-specific alkaline phosphatase (TNALP) or active derivatives thereof.

7. Polyomaviral gene delivery vector particle according to claim 5, wherein the nucleic acid sequence encoding an alkaline phosphatase activity-possessing polypeptide comprises a nucleic acid sequence having at least 70% sequence identity to the amino acid sequence SEQ ID NO: 5 (SPALP).

8. Polyomaviral gene delivery vector particle according to claim 1, wherein the polyomaviral gene delivery vector particle does not comprise a nucleic acid sequence encoding an ectonucleotidase.

9. Polyomaviral gene delivery vector particle according to claim 1, wherein the particle further comprises a nucleic acid sequence encoding a growth factor protein or precursors thereof.

10. Polyomaviral gene delivery vector particle according to claim 9, wherein the particle comprises a first nucleic acid molecule comprising the nucleic acid sequence encoding a phosphatase activity-possessing polypeptide and a second nucleic acid molecule comprising the nucleic acid sequence encoding a growth factor protein or precursors thereof.

11. Polyomaviral gene delivery vector particle according to claim 9, wherein the particle comprises a nucleic acid molecule comprising the nucleic acid sequence encoding a phosphatase activity-possessing polypeptide and the nucleic acid sequence encoding a growth factor protein or precursors thereof.

12. Polyomaviral gene delivery vector particle according to claim 9, wherein the nucleic acid sequence encoding a growth factor protein or precursors thereof comprises a nucleic acid sequence encoding a secreted form of a growth factor protein or precursors thereof.

13. Polyomaviral gene delivery vector particle according to claim 1, wherein the particle further comprises a nucleic acid sequence encoding a growth factor-binding protein or precursors thereof.

14-20. (canceled)

21. Composition comprising an effective amount of polyomaviral gene delivery vector particles according to claim 1.

22. (canceled)

23. A method of treating an inflammatory, degenerative, dystrophic, autoimmune disease and/or allergy in a subject, the method comprising administering to the subject the polyomaviral gene delivery vector particle according to claim 1.

24. A method of removing or inactivating a damage-associated molecular pattern at a site of inflammation in a subject, the method comprising administering to the subject the polyomaviral gene delivery vector particle according to claim 1, thereby removing or inactivating the damage-associated molecular pattern at the site of inflammation in the subject.

25. A method of removing an extracellular adenosine triphosphate (ATP) in a subject, the method comprising administering to the subject the polyomaviral gene delivery vector particle according to claim 1, thereby removing the extracellular ATP in the subject.

26. A method of treating a subject in need of such treatment comprising administering to the subject an effective amount of the polyomaviral gene delivery vector particle according to claim 1.

27. The method of claim 26, wherein the administering comprises administering the polyomaviral gene delivery vector particle by intravenous, intrasynovial, intrapulmonary, intravitreal or subretinal injection.


SEQUENCE LISTING (7 sequences)

SEQ ID NO: 1 - DNA, 1599 bp, Homo sapiens
  source 1..1599: mol_type = genomic DNA
atgcaggggccctgggtgctgctcctgctgggcctgaggctacagctctccctgggcatcatcccagttgaggaggagaacccggacttctggaaccgccaggcagccgaggccctgggtgccgccaagaagctgcagcctgcacagacagccgccaagaacctcatcatcttcctgggtgacgggatgggggtgtctacggtgacagctgccaggatcctaaaagggcagaagaaggacaaactggggcctgagaccttcctggccatggaccgcttcccgtacgtggctctgtccaagacatacagtgtagacaagcatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcaggaaagtcagtgggagtggtaaccaccacacgggtgcagcatgcctcgccagccggcgcctacgcccacacggtgaaccgcaactggtactcggatgccgacgtgcctgcctcggcccgccaggaggggtgccaggacatcgccacgcagctcatctccaacatggacattgatgtgatcctaggtggaggccgaaagtacatgtttcccatggggaccccagaccctgagtacccagatgactacagccaaggtgggaccaggctggacgggaagaatctggtgcaggaatggctggcgaagcaccagggtgcccggtacgtgtggaaccgcactgagctcctgcaggcttccctggacccgtctgtgacccatctcatgggtctctttgagcctggagacatgaaatacgagatccaccgagactccacactggacccctccctgatggagatgacagaggctgccctgctcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttacggagagcgagagcgggagccccgagtatcggcagcagtcagcagtgcccctggacggagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccgcgccggcaccaccgacgccgcgcacccggggccgtccgtggtccccgcgttgcttcctctgctggcagggaccttgctgctgctggggacggccactgctccctga

SEQ ID NO: 2 - DNA, 1608 bp, Homo sapiens
  source 1..1608: mol_type = genomic DNA
atgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtccgtggtccccgcgttgcttcctctgctggccgggaccctgctgctgctggagacggccactgctccctga

SEQ ID NO: 3 - DNA, 1587 bp, Homo sapiens
  source 1..1587: mol_type = genomic DNA
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 4 - DNA, 1575 bp, Homo sapiens
  source 1..1575: mol_type = genomic DNA
atgatttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagtgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcggcaggcagccttgctgcaggccccctgctgctcgcgctggccctctaccccctgagcgtcctgttctga

SEQ ID NO: 5 - DNA, 1503 bp, Homo sapiens
  source 1..1503: mol_type = genomic DNA
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgac

SEQ ID NO: 6 - DNA, 477 bp, Homo sapiens
  source 1..477: mol_type = genomic DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtag

SEQ ID NO: 7 - DNA, 543 bp, Homo sapiens
  source 1..543: mol_type = genomic DNA
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtga